(19) United States
(12) Patent Application Publication
(10) Pub. No.: US 2006/0287884 A1
(43) Pub. Date: Dec. 21, 2006

(54) Pre-delivery drug identification system

(76) Inventors: Neal Joseph Sandy, Middleton, WI (US); Russel Craig Ward, Sun Prairie, WI (US); Ronald P. Makin, Fitchburg, WI (US); William S. Sutherland, Haverhill, MA (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/157,415

(22) Filed: Jun. 21, 2005

(51) Int. Cl. G06Q 10/00; A61K 35/00

(52) U.S. Cl. 705/2; 424/116

(57) ABSTRACT

The present invention is a system and method of pre-delivery drug identification. The system and method is implemented where drugs are being administered, such as in a hospital or clinic, and identifies the drug being administered to the patient before the drug reaches the patient. After identification, the system and method is configured to cross-reference the identified drug with the patient's prescription and allergy information, and to prevent delivery to the patient, if necessary.

### FIELD OF THE INVENTION

The invention relates to the field of drug delivery. More particularly, the invention relates to the field of identifying and confirming drug dosages before administering them to a patient.

### BACKGROUND OF THE INVENTION

Preventable medical errors are becoming increasingly prevalent in the health care industry. Among the biggest problems is adverse drug events or delivering a drug to a patient that should not be given to that patient.

A November 1999 Institute of Medicine report states that “The medication process provides an example where implementing better systems will yield better human performance. Medication errors now occur frequently in hospitals, yet many hospitals are not making use of known systems, nor are they actively pursing new safety systems.”

There has been significant effort in developing systems that are process focused such as bar coding and radiofrequency “tagging” of drugs. Since these methods are process focused, they are inherently prone to error, even though they are an improvement to systems without such processes. Only the identification of the drug itself will insure that the proper drug is being delivered to the patient.

### SUMMARY OF THE INVENTION

The present invention is a system and method of pre-delivery drug identification. The system and method is implemented where drugs are being administered, such as in a hospital or clinic, and identifies the drug being administered to the patient before the drug reaches the patient. After identification, the system and method is configured to cross-reference the identified drug with the patient's prescription and allergy information, and to prevent delivery to the patient, if necessary.

The present invention is a method of pre-delivery drug identification which comprises receiving a drug sample from a drug source into a drug identification unit, identifying the drug sample with the drug identification unit, cross-referencing the identified drug sample with a set of databases, such that the set of databases are configured to confirm the identified drug sample, and preventing the identified drug sample from being administered to a patient with a delivery prevention unit when the set of databases does not confirm the identified drug sample. The drug sample corresponds to a prescription for the patient, and the identifying step further includes identifying the components, the concentration and the dosage of the drug sample. The set of databases may include, but is not limited to a prescription database, an allergy database, and a drug interaction database, and the databases may be central or locally located. The delivery prevention unit is any of the following: a flow valve or an IV line restrictor device, and the drug source is any of a volumetric pump, a syringe pump, or a manual infusion device such as, but not limited to, a bolus syringe.

A system for pre-delivery drug identification which comprises a drug identification unit configured to receive a drug sample from a drug source, wherein the drug identification unit identifies the drug sample, a set of databases coupled to the drug identification unit, configured to allow the drug identification unit to cross-reference the identified drug sample with the set of databases, and a delivery prevention unit coupled to the drug identification unit, the delivery prevention unit configured to prevent the drug sample from being administered to a patient when the set of databases does not confirm the drug sample. The drug sample corresponds to a prescription for the patient, and wherein the drug identification unit is configured to identify the components, the concentration and the dosage of the drug sample. The set of databases may include, but is not limited to a prescription database, an allergy database, and a drug interaction database, and the databases may be centrally or locally located. The delivery prevention unit may include, but is not limited to any of the following: a flow valve or an IV line restrictor device, and the drug source may include, but is not limited to a volumetric pump, a syringe pump, or a manual infusion device such as, but not limited to, a bolus syringe.

### BRIEF DESCRIPTION OF THE DRAWINGS

FIG. 1A-FIG. 1D are block diagrams illustrating embodiments of the system of the present invention.

FIG. 2 is a flow chart illustrating an embodiment of the method of the present invention.

### DETAILED DESCRIPTION OF THE PRESENT INVENTION

The invention is a drug identification system that identifies the drug prior to delivery to a patient. The identification system identifies the compound and/or concentration, cross-references the identification with the prescription, allergies and interactions databases via an interfaced data management system, and has a means to prevent delivery to the patient. Once the appropriate drug is confirmed, the drug compound can be delivered to the patient. The system will record the compound, when it was given and by which clinician. The system would deploy a stop-cock or other line clamping mechanism in order to stop drug flow to the patient prior to actual delivery. The system may or may not have a means to enunciate or otherwise transmit information about the relative correctness of the drug delivery. The system would alert or alarm in a manner that informs the clinician of a potential adverse drug event. The systems will maintain a record of measured drugs and will allow for single or multiple lines to be analyzed.

The primary advantage of the invention is that it will minimize the number of adverse drug events that occur in hospitals, clinics and surgery centers. This advantage may also be translated to home use, emergency and ambulatory use and use in the military. Because the system is able to identify the concentration and/or the compound of the drug itself, it is the most accurate method of drug identification possible. Systems available today rely on processes that have varying levels of compliance and varying levels of propensity for error. And, since the system is dynamic and linked to a drug library via an information system (for example), it can not only detect the drug, but also determine if the drug should be delivered to the patient by comparing the drug to the prescription order as well as to the patient's condition, allergies, or other physiological information, as they are found on the patient's electronic medical record. The ability to automatically stop the delivery of the drug sets the invention apart from other identification systems as well.

Furthermore, the system's ability to determine drug concentrations is highly advantageous and unique. Because the identification system is in-line, yet non-invasive, the system does not waste any drug, and it measures the specific drug sample that is intended for the patient. An alternative embodiment of the present invention implements a split-stream configuration, wherein the sample from the drug source is routed away from the line, is tested, and does not re-enter the line. Finally, drug mixtures (cocktails) would be easily identified and could be disallowed by the system.

It should also be noted that all current drug identification methods are indirect, meaning the drug itself is not identified. This invention actually identifies the drug. Because the identification system can detect specific drug characteristics, it will also recognize mixtures, thereby allowing for the prevention of drug cocktails. All current drug identification methods do not have a means to determine drug concentrations. This invention has such a capability. All current methods cannot stop the inappropriate delivery of a drug. This invention has the ability to automatically stop the delivery of a drug, thereby preventing adverse drug events. The invention can apply to mechanical drug delivery equipment such as IV delivery devices (volumetric or syringe pump) as well as bolus delivery methods. The detection method is non-invasive to the compound which maintains the purity of the drug.

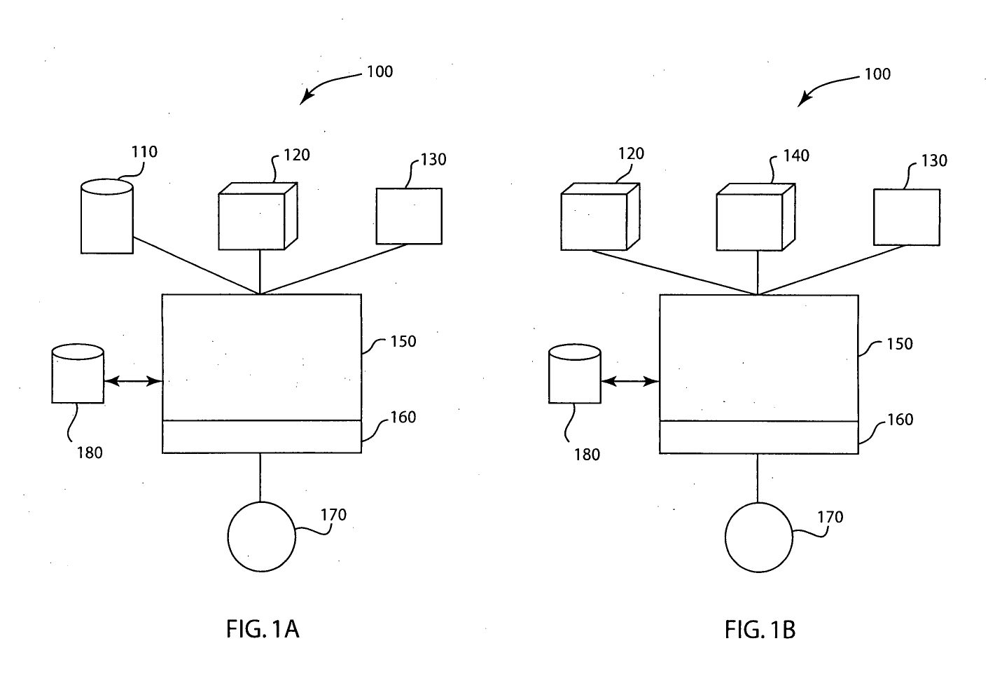

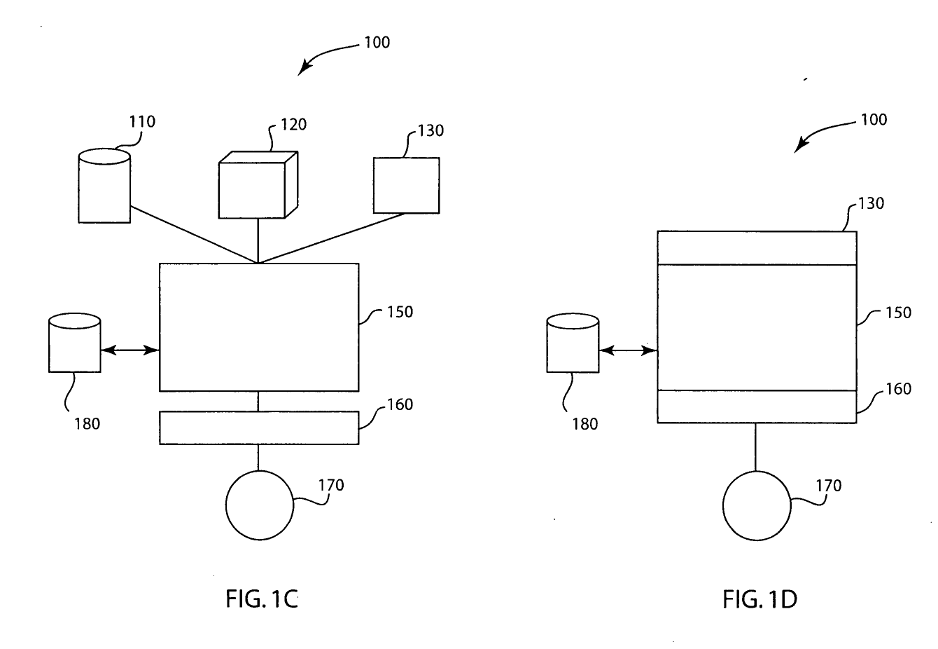

A drug identification system 100 of the present invention is depicted in FIGS. 1A-1D. In FIGS. 1A-1D, the drug identification system 100 includes a drug identification unit 150 configured to receive a sample of a drug to be administered to the patient 170 from a drug source 110, 120, 130, 140. Multiple embodiments of the present invention include various drug sources such as a volumetric pump 110, a syringe pump 120, 140, or a manual infusion device 130, such as, but not limited to a bolus syringe. It should be noted that the drug source 110, 120, 130, 140 utilized in any embodiment of the present invention is likely to be determined by the preference of the physician, the standard practice of the region and/or the particular drug or type of care being administered. Furthermore, referring to FIG. 1B, particular embodiments may utilize more than one particular type of drug source such as a first syringe pump 120 and a second syringe pump 140. As is well known in the art, volumetric pumps 110 and syringe pumps 120, 140 are metered drug delivery sources, while a manual infusion device 130 is not of the metered variety.

Still referring to FIGS. 1A-1D, when a physician prescribes a drug to be administered to a patient 170, one of the aforementioned drug sources 110, 120, 130, 140 is utilized to deliver the drug to the patient 170. The drug identification system 100 of the present invention contemplates receiving a drug sample from the drug source 110, 120, 130, 140 into a drug identification unit 150. The drug identification unit 150 is able to test the drug sample from the drug source 110, 120, 130, 140 and determine a number of characteristics of the drug sample. The drug identification unit 150 is able to identify the element or compound of the drug sample, the concentration of the drug sample, as well as the dosage of the drug sample.

In the preferred embodiment of the present invention, the drug identification unit 150 utilizes raman scattering spectroscopy as a technique to analyze and identify the drug to be administered to the patient 170. The details of raman scattering spectroscopy are included in U.S. Pat. No. 6,868,344 to Nelson. It is further contemplated that alternative embodiments of the present invention can and will include a drug identification unit 150 utilizing other methods known in the art, or future drug identification methods.

The drug identification unit 150 is coupled to a set of patient databases 180. The drug identification unit 150 cross-references the set of patient databases 180 in order to confirm that the drug sample delivered by the drug source 110, 120, 130, 140 is appropriate and safe for the patient 170. The set of databases 180 includes a prescription database, an allergy database and an interaction database, such that the identified drug sample can be cross-referenced against each of these databases 180. Of course, additional databases can be added or subtracted to the set of patient databases 180 as needed or required. Furthermore, the databases may be configured centrally in a network configuration, or locally to the drug identification system 100. In fact, any database 180 configuration known in the art may be used according to the constraints of the particular drug identification system 100.

The drug identification unit 150 will cross-reference the identified drug sample to determine whether the drug sample matches the prescription written by the physician, and will further cross-reference whether the patient 170 is allergic to the drug sample, and further whether the drug sample would interact with any of the patient's 170 other medications. As stated previously, the drug identification unit 150 is configured to identify not only what the drug sample is made up of, but also the concentration and the dosage of the drug sample, thus allowing the drug identification unit 150 to exactly match the drug sample to the prescription.

Still referring to FIGS. 1A-1D, the drug identification system 100 of the present invention also includes a delivery prevention unit 160. If the drug identification unit 150, when cross-referencing the identified drug sample to the set of patient databases 180, cannot confirm that the identified drug sample is appropriate and/or safe to administer to the patient 170, the delivery prevention unit 160 will prevent the drug sources 110, 120, 130, 140 from administering the drug to the patient 170. Preferably, the delivery prevention unit 160 is an electronically controlled flow valve, but can also be any IV flow restrictor known or later developed in the art. Only when the drug identification unit 150 is able to cross-reference the set of patient databases 180, and confirm that the drug sample exactly matches the prescription, and is not harmful to the patient, will the drug identification unit prompt the delivery prevention unit 160 to allow the drug to be administered to the patient 170. As is shown in FIGS. 1A, 1B and 1D, preferably the delivery prevention unit 160 is directly coupled to the drug identification unit and is likely implemented with the drug identification unit. Other embodiments, such as that depicted FIG. 1C, will include a delivery prevention unit 160 separate yet coupled to the drug identification unit 150. Referring to FIG. 1D, the drug identification system 100 of the present invention may also be implemented in one unit, including a manual infusion device 130, a drug identification unit 150 and a delivery prevention unit 160.

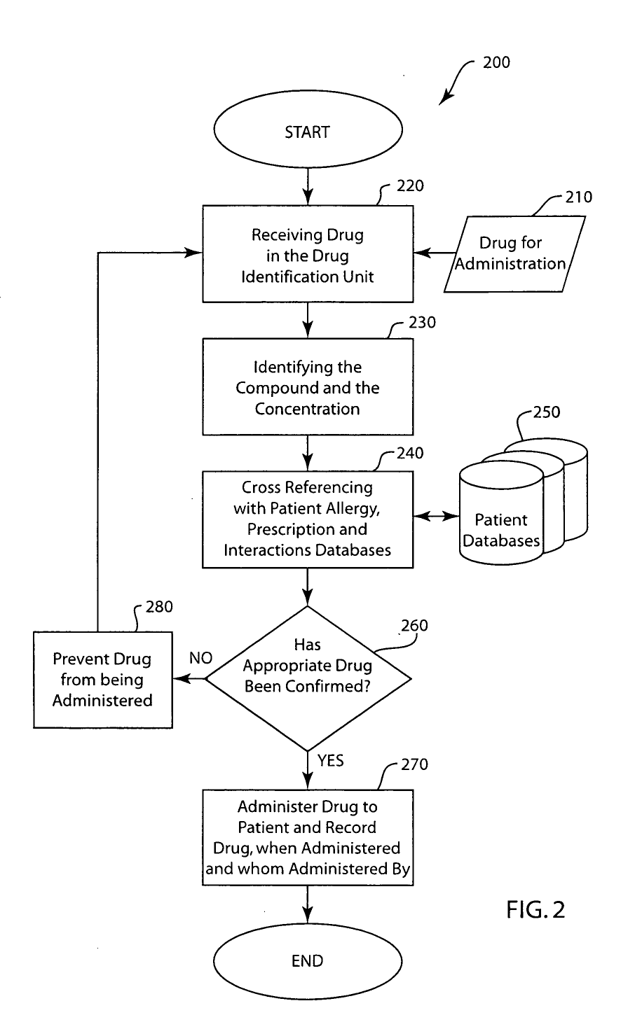

Referring now to FIG. 2, a drug identification method 200 of the present invention is depicted. In step 220, the drug for administration 210 to the patient is received in the drug identification unit. In step 230, the drug is identified, including the actual compound, the concentration and the dosage of the drug for administration 210. In step 240, the identified drug is cross-referenced with a set of patient databases 250 to confirm whether the identified drug matches the prescription written by the physician. In step 240, the identified drug is also cross-referenced to the patient databases 150 to determine whether the identified drug will cause problems with the patients due to allergies and/or interactions with other drugs. In step 260, if the identified drug is not confirmed as the proper prescription or being dangerous to the patient, then in step 280 the identified drug is prevented from being administered to the patient and the method 200 starts again at step 220. However, if the identified drug is confirmed to be appropriate for administration to the patient, then the drug is administered to the patient in step 270. Furthermore, in step 270, the administration of the identified drug is recorded, and the time and the person who administered the drug is recorded as well.

The present invention has been described in terms of specific embodiments and incorporated details to facilitate the understanding of the principles of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

What is claimed is:

1. A method of pre-delivery drug identification, the method comprising:
a.) receiving a drug sample from a drug source into a drug identification unit;
b.) identifying the drug sample with the drug identification unit;
c.) cross-referencing the identified drug sample with a set of databases, such that the set of databases are configured to confirm the identified drug sample; and
d.) preventing the identified drug sample from being administered to a patient with a delivery prevention unit when the set of databases does not confirm the identified drug sample.

2. The method according to claim 1, wherein the drug sample corresponds to a prescription for the patient.

3. The method according to claim 1, wherein the identifying step further includes identifying the components, the concentration and the dosage of the drug sample.

4. The method according to claim 1, wherein the set of databases includes:
a.) a prescription database;
b.) an allergy database; and
c.) a drug interaction database.

5. The method according to claim 1, wherein the delivery prevention unit is any of the following:
a.) a flow valve; and
b.) an IV line restrictor device.

6. The method according to claim 1, wherein the drug source is a volumetric pump.

7. The method according to claim 1, wherein the drug source is a syringe pump.

8. The method according to claim 1, wherein the drug source is a manual infusion device.

9. A system for pre-delivery drug identification, the system comprising:
a.) a drug identification unit configured to receive a drug sample from a drug source, wherein the drug identification unit identifies the drug sample;
b.) a set of databases coupled to the drug identification unit, configured to allow the drug identification unit to cross-reference the identified drug sample with the set of databases; and
c.) a delivery prevention unit coupled to the drug identification unit, the delivery prevention unit configured to prevent the drug sample from being administered to a patient when the set of databases does not confirm the drug sample.

10. The system according to claim 9, wherein the drug sample corresponds to a prescription for the patient.

11. The system according to claim 9, wherein the drug identification unit is configured to identify the components, the concentration and the dosage of the drug sample.

12. The system according to claim 9, wherein the set of databases includes:
a.) a prescription database;
b.) an allergy database; and
c.) a drug interaction database.

13. The system according to claim 9, wherein the delivery prevention unit is any of the following:
a.) a flow valve; and
b.) an IV line restrictor device.

14. The system according to claim 9, wherein the drug source is a volumetric pump.

15. The system according to claim 9, wherein the drug source is a syringe pump.

16. The system according to claim 9, wherein the drug source is a manual infusion device.

17. A method of pre-delivery drug identification of a drug sample, the drug sample corresponding to a prescription of a patient, the method comprising:
a.) receiving the drug sample into a drug identification unit;
b.) identifying the components, the concentration and the dosage of the drug sample with the drug identification unit;
c.) cross-referencing the identified drug sample with a prescription database, an allergy database and a drug interaction database, such that the databases are configured to confirm the identified drug sample; and
d.) preventing the identified drug sample from being administered to a patient with a delivery prevention unit when any one of the databases does not confirm the identified drug sample.
